(19) United States
(12) Patent Application Publication
(10) Pub. No.: US 2010/0082018 A1
(43) Pub. Date: Apr. 1, 2010

(54) METHOD AND SYSTEM FOR RESHAPING THE CORNEA

(76) Inventors: Daryus Panthakey, London (GB); Mathias Glasmacher, Reilingen (DE); Frieder Loesel, Mannheim (DE)

Correspondence Address: NYDEGGER & ASSOCIATES, 348 OLIVE STREET, SAN DIEGO, CA, 92103, US

(21) Appl. No.: 12/239,443

(22) Filed: Sep. 26, 2008

(51) Int. Cl. A61F 9/01

(52) U.S. Cl. 606/5

(57) ABSTRACT

A system and method are provided for reshaping the surface of a resilient transparent material such as a cornea. In the system, a laser unit generates a femto-second laser beam to deliver focused energy inside the material. Specifically, the energy is focused over a defined spot pattern to weaken the material. Further, the system includes a contact element that forms a contour surface. In order to reshape the material, the system provides for holding the contour surface of the contact element against the surface of the weakened material. After holding the contour surface against the material for a pre-determined time duration, the surface of the material is reshaped with a desired configuration that substantially mimics the contour surface of the contact element.

### FIELD OF THE INVENTION

The present invention pertains generally to systems for delivering focused energy inside a resilient transparent material. More particularly, the present invention pertains to a laser system for delivering radiation to weaken a material, and to a contact element for holding the material in a desired configuration as the material sets after the laser procedure. The present invention is particularly, but not exclusively, useful for reshaping the surface of the resilient transparent material to improve the optical performance of the resilient transparent material.

### BACKGROUND OF THE INVENTION

There are many laser procedures in which it is desirable to modify the optical performance of a resilient transparent material. Typically, these procedures require a reconfiguration of the material. For a variety of laser procedures, the desired results are obtained immediately and no further operations or treatments are required. For some laser procedures, however, the corneal stromal tissue may not heal in an optimal manner without further treatment. In fact, due to the corneal stromal tissue's internal structure and its biomechanical properties (e.g. intraocular pressure, and inherent biomechanical stresses and strains), the cornea may regress to a non-corrected state during healing after surgery.

In view of the complexity of the cornea's internal structure and its related biomechanical properties, it is not surprising that surgical procedures alone may not resolve every optical aberration. However, with the knowledge of the stromal tissue's biomechanical properties, the behavior of the stromal tissue in response to a laser procedure may be predicted. Further, it has been appreciated that the behavior of the stromal tissue in response to a laser procedure may be controlled through the application of additional treatment after the laser procedure. Specifically, the external application of a specifically designed contact element, such as a lens, after a laser procedure allows a definitive prediction of the dynamic change in the shape of a cornea after treatment. Structurally, the contact lens can mechanically compel the cornea to set in a desired configuration during the post-surgery healing process. As a result, the use of such a contact lens subsequent to laser surgery can achieve greater optical changes than the use of laser procedures alone.

In light of the above, it is an object of the present invention to provide systems and methods for permanently reshaping a surface of a resilient transparent material such as a cornea. Another object of the present invention is to provide systems and methods for disrupting stress distributions within a cornea and for thereafter applying a mechanical force to the surface of the cornea. It is yet another object of the present invention to provide a contact lens for use subsequent to a laser procedure to apply a desired mechanical force to the surface of the cornea. It is yet another object of the present invention to provide systems and methods for permanently reshaping the surface of a cornea with a desired configuration that corresponds to the contour surface of a contact lens. Another object of the present invention is to provide systems and methods for reshaping the surface of a cornea in which contact lenses having unique contour surfaces are sequentially applied to the surface of the cornea to control the cornea's response to a laser procedure. Still another object of the present invention is to provide systems and methods for reshaping the surface of a cornea that are easy to use and comparatively cost effective.

### SUMMARY OF THE INVENTION

The present invention is directed to a system and method for reshaping the surface of a resilient transparent material, such as the cornea of an eye. For the present invention, the system includes a laser unit for delivering focused energy inside the material. The focused energy weakens the material (e.g., the cornea) and causes it to reconfigure in response to subsequent forces on the material. Preferably, the laser unit generates a femto-second laser beam.

Also, the system of the present invention includes either a single contact lens, or a plurality of contact lenses for sequential use. Specifically, each contact lens has a unique contour surface. And, structurally, the contour surfaces of the contact lenses can be graduated from an initial contour surface to a final desired contour surface. In practice, the final desired contour surface is shaped to conform the anterior surface of the cornea to a desired radius of curvature.

For the present invention, the system further includes an element for holding the contour surface of a selected contact lens against the surface of the cornea, after a laser procedure has been performed. While the holding element may include a mechanical apparatus such as a suction ring with a holder, more often it will simply be the patient's eyelid. Typically, in use, each contact lens will be held against the cornea for a pre-determined time duration (e.g., eight hours). As each contact lens is held against the surface of the material, in sequence, for a respective time duration, the material becomes slightly reconfigured. Eventually, with the last contact lens in the sequence, the corneal surface is shaped into the desired configuration.

In accordance with the present invention, the system also includes a mechanism for applying a softening agent to the cornea. Preferably, the softening agent includes enzymes and can be applied before the initial contact lens is applied to the eye, while a contact lens is held to the cornea, and/or after a contact lens is removed and before the next contact lens is applied. Further, the system includes a device for applying a curing agent to the cornea. For instance, the applicator device may be a dropper that coats the corneal surface with a curing agent, such as Riboflavin, that will penetrate the corneal surface. Additionally, the system is provided with a unit for radiating UV light onto the coated surface and into the material to interact with the curing agent. Importantly, through this interaction the curing agent and UV light cross-link and stiffen the stromal tissue in the cornea. Preferably, UV light is radiated into the cornea after the final contact lens has been held against the cornea for the respective pre-determined time duration.

Before use of the laser unit in the present invention, the cornea is measured for a refractive error. As a result, diagnostic data is received regarding the refractive error. Thereafter, the diagnostic data is inputted into a mathematical model to obtain geometrical parameters for the laser procedure. Specifically, these geometrical parameters define a spot pattern, or a series of spot patterns, that are to be performed during the laser procedure. As a result of the laser procedure, the cornea is weakened by the disruption of intrastromal stress distributions. As noted above, however, the corneal configuration may change due to biomechanical forces in the eye after the laser procedure. Therefore, the cornea must somehow be constrained, or guided, to its eventual desired configuration. In order to ensure that the weakened cornea responds to the laser procedure as required for vision correction, each successive contact lens provides a known mechanical force to the corneal surface. In this manner, the corneal surface is reshaped to the desired configuration.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified, schematic diagram showing the components of the system for reshaping the cornea in accordance with the present invention;

FIG. 2 is a schematic illustrating the contour surfaces of the sequence of contact lenses for use in the system of FIG. 1; and

FIG. 3 is an operational flow chart illustrating the steps of the method in accordance with the present invention.

### DESCRIPTION OF THE PREFERRED EMBODIMENTS

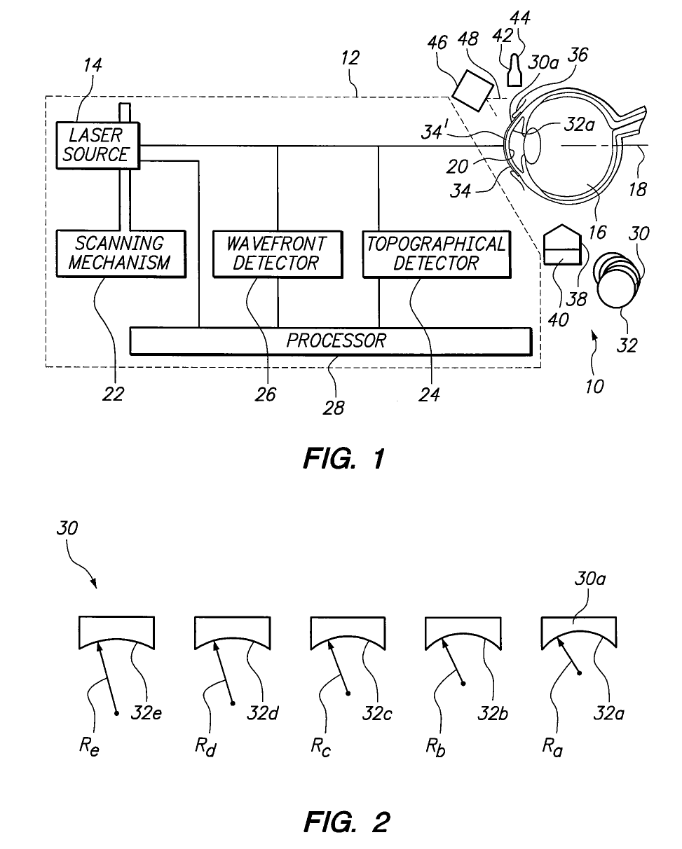

Referring initially to FIG. 1, a system in accordance with the present invention is shown schematically and generally designated 10. As shown, the system 10 includes a laser unit 12 provided with a laser source 14 to generate a laser beam. Further, the laser source 14 is positioned relative to the eye 16 to allow a laser beam to be directed along a beam path that is collinear with the optical axis 18 of the eye 16. For the present invention, the laser unit 12 may treat the corneal tissue 20 by means of ablation, disruption, chemical decomposition and/or combinations thereof. In certain embodiments, to perform a laser procedure to treat corneal tissue 20, the laser source 14 may have a mode in which the laser source 14 generates a continuous train of ultra-short pulses, with each pulse having a pulse duration in a femto-second range. For laser procedures involving intrastromal photoablation of corneal tissue, e.g., laser induced optical breakdown, each pulse will have an energy level that is above the threshold necessary for the photoablation of stromal tissue (e.g., above approximately one and one half microjoules per ten micron diameter spot size). Alternatively, corneal tissue 20 may be treated with several sub-threshold pulses to obtain an accumulated effect. While photoablation and accumulated sub-threshold pulse treatment are noted here, other types of laser procedures may be performed by the laser unit 12 to treat the cornea 20.

Continuing now with reference to FIG. 1, it can be seen that the laser unit 12 further includes a scanning mechanism 22 for moving the focus of the laser beam relative to the eye 16. Specifically, the focus of the laser beam is moved along a pre-determined path in the cornea 20. As further shown in FIG. 1, the laser unit 12 can also include a topography detector 24 that is capable of determining the topographic properties within corneal tissue 20. Further, FIG. 1 shows that the laser unit 12 can include a wavefront detector 26, such as a Hartmann-Shack sensor, which is capable of modeling a wavefront. Additionally, as shown, the laser unit 12 includes a processor 28 which is preferably a dedicated computer. The processor 28 is provided to process data and control the other components of the laser unit 12 including the scanning mechanism 22.

FIG. 1 also shows that the system 10 includes a plurality of contact elements 30, such as contact lenses, for successive use after the laser procedure is performed. In FIG. 1, the final contact lens 30a is shown in contact with the eye 16. Structurally, each contact lens 30 includes a unique posterior contour surface 32. After the laser procedure is performed by the laser unit 12, each lens 30 in the plurality of contact lenses 30 is successively applied to the eye 16 to reshape its anterior surface 34. Importantly, the desired contour surface 32a conforms the anterior surface 34 of the eye 16 to the desired configuration 34′ shown in FIG. 1.

As shown in FIG. 2, the contour surfaces 32 of the contacts lenses 30 are graduated from an initial contour surface 32e through intermediate contour surfaces 32d, 32c, and 32b, to the desired contour surface 32a of the final contact lens 30a. As illustrated, each contour surface 32 is flatter than the subsequent contour surface 32. Specifically, each contour surface 32a-32e has a respective radius of curvature Rₐ-Rₑ, with Rₐ<R_{b}<R_{c}<R_{d}<Rₑ. The use of a sequence of contact lenses 30 ending with contact lens 30a would cause the anterior surface 34 of the cornea 20 to become more convex during treatment. Of course, the sequence of contact lenses 30 can be used in reverse order to cause the anterior surface 34 of the cornea 20 to become flatter during treatment.

As shown in FIG. 1, the system 10 further includes an element 36 for holding each contact lens 30 against the anterior surface 34 of the eye 16. In FIG. 1, this element 36 is the patient's eyelids, however, it is envisioned that a mechanical apparatus, such as a suction ring with a holder, may be used for this purpose. Further, while the contact lens 30 may be held directly against the anterior surface 34 of the eye 16, it is envisioned that the contact lens 30 may indirectly contact the eye 16, i.e., the lens 30 may be held against the outer surface of the eyelid. As further shown, the system 10 includes a mechanism 38 for applying a softening agent 40 to the eye 16. In FIG. 1, the mechanism 38 is a fluid container which holds the softening agent 40 which typically will include enzymes. The softening agent 40 may be applied to the eye 16 by squeezing or pouring the agent 40 from the container 38. Also, the system 10 includes a device 42, such as a dropper, for applying a curing agent 44 to the eye 16. Preferably, the curing agent 44 is Riboflavin, which will penetrate the surface 34 of the eye 16 upon application. In conjunction with the device 42, the system 10 includes a unit 46 for radiating UV light 48 onto the coated surface 34 and into the eye 16 to cross-link and stiffen the corneal tissue 20.

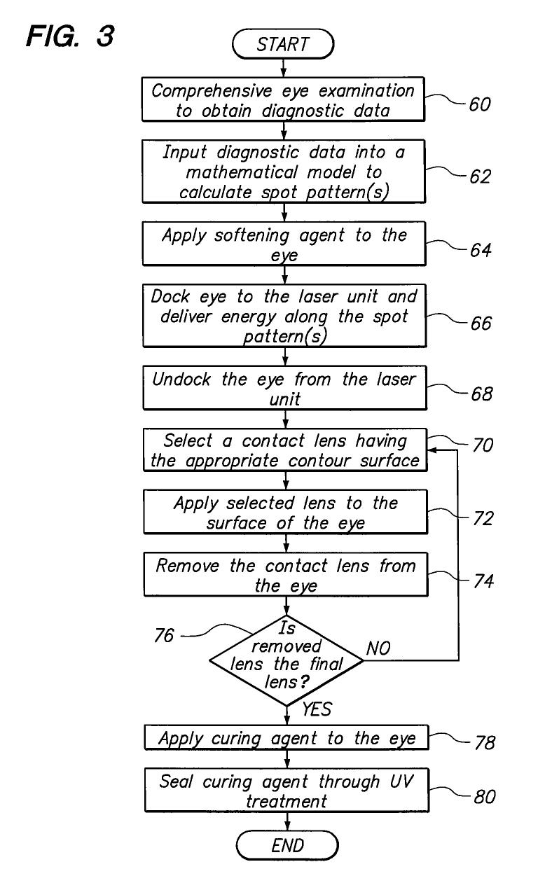

Referring to FIG. 3 (with cross-reference to FIG. 1), the method for reshaping the surface 34 of a resilient transparent material like a cornea 20 is illustrated. As shown, the method begins at action block 60 where a comprehensive eye examination is performed on a patient to obtain diagnostic data. Then, at action block 62, the diagnostic data is inputted into a mathematical model that calculates the spot pattern or patterns necessary to correct or optimize the patient's vision.

After the necessary spot patterns are calculated, and before any laser treatment, the softening agent 40 (action block 64) may be applied to the surface 34 of the eye 16. Thereafter, the eye 16 is docked to the laser unit 12 and focused energy is delivered by the laser unit 12 over the spot patterns inside the corneal tissue 20 of the eye 16 (action block 66). As a result, the corneal tissue 20 of the eye 16 is weakened, and intracorneal stress distributions may be disrupted. After the laser procedure is completed, the eye 16 is undocked from the laser unit 12 (action block 68). At this point, the eye 16 is ready to be reshaped by the use of the contact lenses 30. Specifically, at action block 70, a contact lens 30 having the appropriate contour surface 32 is selected from the plurality of contact lenses 30. For instance, for the correction of an eye 16 that requires an anterior surface 34 with a longer radius of curvature, i.e., a flatter surface 34, the initial contact lens 30 will have a contour surface 32 with the shortest radius of curvature among the plurality of lenses 30. Accordingly, the final contact lens 30a will have a contour surface 32a with the longest radius of curvature among the plurality of lenses 30.

At action block 72, the selected contact lens 30 is applied and held against the surface 34 of the eye 16 for a pre-determined duration of time, e.g., eight hours, sufficient to ensure that the healing processes in the eye 16 have stabilized suitably to prevent regression to the eye's original state. As indicated above, the contact lens 30 may be held against the surface 34 of the eye 16 by the patient's eyelids 36 or by a device. After the pre-determined duration of time, the selected contact lens 30 is removed from the eye 16 at action block 74. Thereafter, it is determined whether the removed contact lens 30 is the final contact lens 30a (inquiry block 76). If it is not the final contact lens 30a, then the method may resume at action block 70 with the selection of the next contact lens 30 in the succession of contact lenses 30.

When it is determined, at inquiry block 76, that the final contact lens 30a is the selected lens 30 that has been removed from the eye 16 at action block 74, then the curing agent 44 is applied to the surface 34 of the eye 16 (action block 78). Thereafter, the curing agent 44 is sealed (action block 80). Specifically, UV light 48 is radiated by the unit 46 into the corneal tissue 20 of the eye 16 in order to cross-link and stiffen the corneal tissue 20. Importantly, the use of a succession of contact lenses 30 provides a staged treatment sequence to control the healing process and permanently influence the retention of the desired corneal configuration 34′, i.e., for a pre-determined period of time such as at least one year.

While the particular Method and System for Reshaping the Cornea as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for reshaping a surface of a resilient transparent material which comprises:
a laser unit for delivering focused energy over at least one defined spot pattern inside the material, to weaken the material;
a contact element having a contour surface; and
a means for holding the contour surface of the contact element against the surface of the weakened material for a pre-determined time duration to reshape the surface of the material with a desired configuration, wherein the desired configuration substantially mimics the contour surface of the contact element.

2. A system as recited in claim 1 further comprising a plurality of contact elements wherein each contact element is held against the surface of the material, in sequence, for a respective time duration, and wherein the last contact element in the sequence is formed with the contour surface for reshaping the surface of the material to the desired configuration.

3. A system as recited in claim 1 wherein the pre-determined time duration is approximately eight hours.

4. A system as recited in claim 1 wherein the spot pattern is calculated from diagnostic data.

5. A system as recited in claim 1 wherein the contour surface of the contact element is held directly against the surface of the weakened material.

6. A system as recited in claim 1 further comprising a means for applying a softening agent to the material, wherein the softening agent comprises enzymes.

7. A system as recited in claim 1 further comprising a means for applying a curing agent to the material.

8. A system as recited in claim 7 wherein the means for applying the curing agent comprises:
a means for coating the surface of the material with Riboflavin, wherein the Riboflavin penetrates the surface of the material; and
a unit for radiating UV light onto the coated surface and into the material to cross-link and stiffen the material.

9. A system as recited in claim 1 wherein the resilient transparent material is a patient's cornea.

10. A system as recited in claim 1 wherein the holding means is a patient's eyelid.

11. A system for reshaping a surface of a resilient transparent material which comprises:
a laser unit for delivering focused energy over at least one defined spot pattern inside the material to weaken the material; and
a means for pressing a contour surface against the surface of the weakened material for a pre-determined time duration to reshape the surface of the material with a desired configuration, wherein the desired configuration substantially mimics the contour surface.

12. A system as recited in claim 11 wherein the pressing means utilizes a plurality of contact elements wherein each contact element is held against the surface of the material, in sequence, for a respective time duration, and wherein the last contact element in the sequence is formed with the contour surface for reshaping the surface of the material to the desired configuration.

13. A system as recited in claim 11 wherein the spot pattern is calculated from diagnostic data.

14. A system as recited in claim 11 further comprising a means for applying a softening agent comprising enzymes to the material.

15. A system as recited in claim 11 further comprising a means for applying a curing agent to the material.

16. A system as recited in claim 11 wherein the resilient transparent material is a patient's cornea, and wherein the pressing means comprises a contact element forming the contour surface, wherein the contact element is held against the surface of the cornea by the patient's eyelid.

17. A method for reshaping a surface of a resilient transparent material which comprises the steps of:
measuring a characteristic of the transparent material to obtain diagnostic data;
calculating a spot pattern from the diagnostic data;
delivering focused energy over the spot pattern inside the material, to weaken the material; and
holding a contour surface against the surface of the weakened material for a pre-determined time duration to reshape the surface of the material with a desired configuration, wherein the desired configuration substantially mimics the contour surface.

18. A method as recited in claim 17 wherein the contour surface is formed by a contact element, wherein the transparent material is a patient's cornea, and wherein the holding step is accomplished by pressing the contact element onto the surface of the cornea with the patient's eyelid.

19. A method as recited in claim 18 wherein the contact element is selected from a plurality of contact elements, and wherein the holding step is accomplished by pressing each contact element against the surface of the material, in sequence, for a respective time duration, and wherein the last contact element in the sequence is formed with the contour surface for reshaping the surface of the material to the desired configuration.

20. A method as recited in claim 19 further comprising the step of applying a softening agent to the material before the delivering step

21. A method as recited in claim 19 further comprising the step of applying a curing agent to the material after the delivering step.
